(19) United States
(12) Patent Application Publication
(10) Pub. No.: US 2024/0374961 A1
(43) Pub. Date: Nov. 14, 2024

(54) RECOMMENDED EXERCISE PRESENTATION DEVICE, SERVER DEVICE, EXERCISE THERAPY SUPPORT SYSTEM, RECOMMENDED EXERCISE PRESENTATION METHOD, AND CONTROL PROGRAM

(71) Applicant: KYOCERA Corporation, Kyoto-shi, Kyoto (JP)

(72) Inventor: Koji ONISHI, Kyoto-shi, Kyoto (JP)

(21) Appl. No.: 18/691,503

(22) PCT Filed: Sep. 6, 2022
(86) PCT No.: PCT/JP2022/033423

§ 371 Date: Mar. 13, 2024

(30) Foreign Application Priority Data
Sep. 14, 2021 (JP) 2021-149585

(51) Int. Cl. A63B 24/00; A61B 5/00; A61B 5/11; G16H 20/30

(52) U.S. Cl. CPC A63B 24/0075; A61B 5/11; A61B 5/4824; G16H 20/30

(57) ABSTRACT

An exercise therapy suitable for each patient is presented. A recommended exercise presentation device includes a pain information acquisition unit and a presentation unit. The pain information acquisition unit acquires first pain information about pain in a target site of a user. The presentation unit presents, to a user, recommended exercise for the user, as determined based on the first pain information.

### TECHNICAL FIELD

The present disclosure relates to a recommended exercise presentation device and the like for presenting recommended exercise to patients.

### BACKGROUND OF INVENTION

It is known that exercise therapy for causing a patient to perform predetermined exercise is effective for treatment of physical disorders and diseases, improvement of symptoms, and the like.

Patent Document 1 discloses an exercise therapy device capable of appropriately adjusting a load.

### CITATION LIST

### Patent Literature

Patent Document 1: WO 2020/026440

### SUMMARY

### Solution to Problem

A recommended exercise presentation device according to an aspect of the present disclosure includes a pain information acquisition unit that acquires first pain information about pain in a target site of a user, and a presentation unit that presents, to the user, recommended exercise for the user, as determined based on the first pain information.

A server device according to an aspect of the present disclosure is a server device that can communicate with the recommended exercise presentation device according to an aspect of the present disclosure and a communication terminal used by a healthcare professional corresponding to at least one user of the recommended exercise presentation device and includes a period setting reception unit that causes, from the communication terminal of the healthcare professional, an exercise period where the at least one user is caused to perform the recommended exercise to be input, and a user information acquisition unit that acquires user information about the at least one user from the recommended exercise presentation device, in which the user information includes effect information including at least one selected from the group consisting of the first pain information, information about the recommended exercise presented by the recommended exercise presentation device, second pain information about pain in the target site of the at least one user who has performed the recommended exercise for a first period, and exercise history information indicating a history of the recommended exercise that has performed by the at least one user.

An exercise therapy support system according to an aspect of the present disclosure includes the recommended exercise presentation device according to an aspect of the present disclosure, a communication terminal used by a healthcare professional corresponding to a user of the recommended exercise presentation device, and the server device according to an aspect of the present disclosure.

An exercise therapy support system according to an aspect of the present disclosure includes the recommended exercise presentation device according to an aspect of the present disclosure, a motion detection device that acquires detection information about a motion of the target site of the user, a communication terminal used by a healthcare professional corresponding to a user of the recommended exercise presentation device, and the server device according to an aspect of the present disclosure.

A recommended exercise presentation method according to an aspect of the present disclosure includes acquiring first pain information about pain in a target site of a user, and presenting, to the user, recommended exercise for the user, as determined based on the first pain information.

A control program according to an aspect of the present disclosure is a control program for causing a computer to function as the recommended exercise presentation device according to an aspect of the present disclosure, and for causing the computer to function as the pain information acquisition unit and the presentation unit.

A control program according to an aspect of the present disclosure is a control program for causing a computer to function as the server device according to an aspect of the present disclosure, and for causing the computer to function as the period setting reception unit and the user information acquisition unit.

### BRIEF DESCRIPTION OF THE DRAWINGS

FIG. 1 is a diagram illustrating an example of a configuration of an exercise therapy support system according to an embodiment.

FIG. 2 is a functional block diagram illustrating an example of a configuration of a server device.

FIG. 3 is a diagram illustrating an example of a data structure of a registration information DB.

FIG. 4 is a diagram illustrating an example of a data structure of a recommended exercise DB.

FIG. 5 is a diagram illustrating an example of a data structure of a web page DB.

FIG. 6 is a functional block diagram illustrating an example of a configuration of a recommended exercise presentation device.

FIG. 7 is a functional block diagram illustrating an example of a configuration of a communication terminal.

FIG. 8 is a sequence diagram illustrating an example of the flow of processing performed by the exercise therapy support system.

FIG. 9 is a diagram illustrating an example of a configuration of the exercise therapy support system including a motion detection device.

### DESCRIPTION OF EMBODIMENTS

When an exercise therapy is instructed to a patient, it is considered as important to advise every patient to perform exercise suitable for the patient.

According to an aspect of the present disclosure, an exercise therapy suitable for each patient can be presented.

### First Embodiment

An embodiment of the present disclosure will be described in detail below.

In the present disclosure, an “exercise therapy” is a therapy in which a patient (user) performs exercise to treat a disorder, a disease, or the like in a target site of the patient. An “exercise therapy” may include utilizing exercise to reduce the occurrence of disorders, diseases, and the like in a target site and/or to maintain or further improve the state of a target site after treatment. Examples of exercise included in the exercise therapy include aerobic exercise, anaerobic exercise, muscle training, and stretching.

In the present disclosure, the “target site” means a movable part of a body, and may be, for example, an elbow, a shoulder, a wrist, a neck, a torso, a waist, a knee, an ankle, or the like. The “target site” may be a knee joint, a shoulder joint, a hip joint, or the like.

Examples of target disease to which an exercise therapy is applied (i.e., prescribed) include orthopedic diseases, lifestyle-related diseases, and medical diseases. Orthopedic disorders may include a variety of disorders that occur in spines and joints such as knees. Orthopedic diseases may also include bone fractures, lower back pain, knee pain, shoulder pain, and hip pain. Lifestyle-related diseases may include diabetes, hypertension, and dyslipidemia. Medical diseases may include circulatory diseases and respiratory diseases. For example, an exercise therapy for circulatory diseases and an exercise therapy for respiratory diseases may be included in cardiac rehabilitation and respiratory rehabilitation, respectively.

### Configuration of Exercise Therapy Support System 100

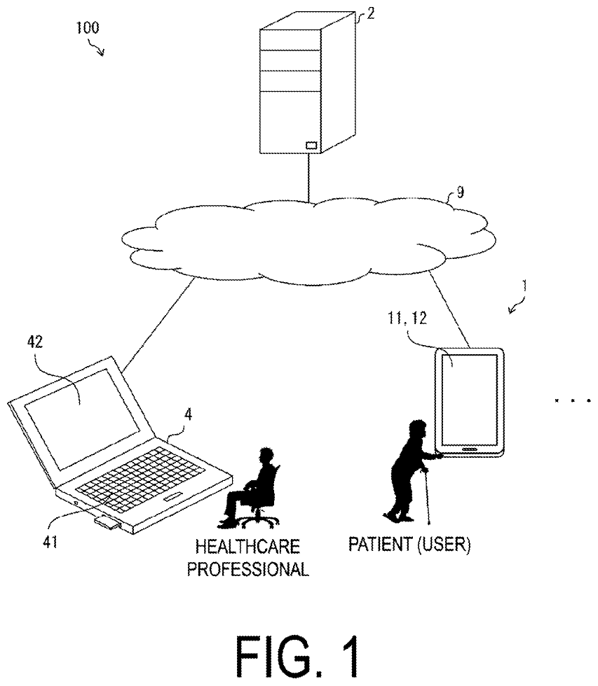

First, a configuration of an exercise therapy support system 100 according to an embodiment of the present disclosure will be described using FIG. 1. FIG. 1 is a diagram illustrating an example of a schematic configuration of the exercise therapy support system 100 according to an embodiment.

The exercise therapy support system 100 is a system capable of presenting exercise suitable for each patient and supporting the exercise therapy of the patient. To treat a target site, the patient is provided with a predetermined application program (a recommended exercise presentation application 131) related to drugs (or supplements), exercise instructions, and exercise therapies from healthcare professionals. The recommended exercise presentation application 131 is installed in a computer such as a smartphone used by the patient. The computer functions as a recommended exercise presentation device 1 to be described below by activating the installed recommended exercise presentation application 131. In the present disclosure, the number of patients may be at least one.

When a patient performs an exercise therapy at home, the recommended exercise presentation device 1 can present the patient with a recommended exercise suitable for the patient or provide various kinds of information about the recommended exercise. In addition, the recommended exercise presentation device 1 can manage records related to the recommended exercise performed by the patient.

On the other hand, a healthcare professional who uses the exercise therapy support system 100 is provided with a predetermined application program (a recommended exercise presentation application 431). The recommended exercise presentation application 431 is installed in a computer used by the healthcare professional. The computer functions as a communication terminal 4 to be described below by activating the installed recommended exercise presentation application 431.

By using the communication terminal 4, the healthcare professional can set an exercise period during which the patient is caused to perform the recommended exercise and can check the history of the recommended exercise performed by the patient, or the like.

The exercise therapy support system 100 includes a server device 2, the recommended exercise presentation device 1 used by a patient, and the communication terminal 4 used by a healthcare professional.

In the exercise therapy support system 100, the server device 2, the recommended exercise presentation device 1, and the communication terminal 4 may be communicatively connected to each other. For example, the server device 2 may be communicatively connected to the recommended exercise presentation device 1 and the communication terminal 4 via a communication network 9 as illustrated in FIG. 1. The recommended exercise presentation device 1 and the server device 2, and the communication terminal 4 and the server device 2 may be connected by wireless communication or may be connected by wired communication.

The exercise therapy support system 100 may be configured to include a plurality of recommended exercise presentation devices 1 and a plurality of communication terminals 4. That is, the exercise therapy support system 100 can also present each patient examined by each of a plurality of healthcare professionals with recommended exercise suitable for the patient.

Hereinafter, the server device 2, the recommended exercise presentation device 1, and the communication terminal 4 will be described.

### Server Device 2

The server device 2 is a computer that generates web pages related to an exercise therapy for each patient and manages the web pages. Any known hardware and operating system (OS) of a computer are applicable. A web page generated by the server device 2 may be browsed only by the patient himself/herself and the healthcare professional corresponding to the patient.

This facilitates sharing of information about the exercise therapy between the patient himself/herself and the healthcare professional corresponding to the patient.

The server device 2 may be implemented by a cloud computing system.

The server device 2 may have a function of determining recommended exercise suitable for each patient based on first pain information acquired from the recommended exercise presentation device 1. Here, the first pain information is information about the pain of the patient in the target site. Information about pain may be, for example, the degree of pain (including the presence or absence of pain), the type of the pain, or a combination thereof. The first pain information may be information about the intensity of the pain felt by the patient himself/herself at the target site before the exercise therapy presented by the exercise therapy support system 100 is started (e.g., when the patient receives a medical examination by a presenting healthcare professional).

The server device 2 may have a function of acquiring an exercise period in which a patient is caused to perform recommended exercise set by a healthcare professional, managing the start and end of the exercise period, and monitoring the execution of the exercise therapy by the patient during the exercise period. For example, at the end of the exercise period, the server device 2 may transmit a first completion report indicating that the exercise period has ended to the communication terminal 4 of the healthcare professional.

Each time a patient has performed the recommended exercise, the server device 2 may transmit a report to that effect to the communication terminal 4 of the healthcare professional. Alternatively, when the period in which the patient does not perform the recommended exercise is equal to or longer than the second period, the server device 2 may transmit an exercise suspension report to that effect to the communication terminal 4 of the healthcare professional.

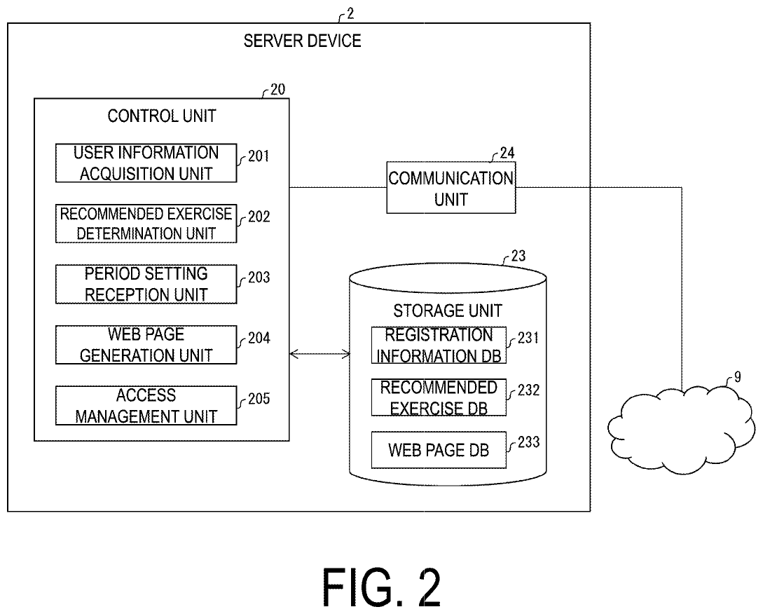

A configuration of the server device 2 will be described using FIG. 2 with reference

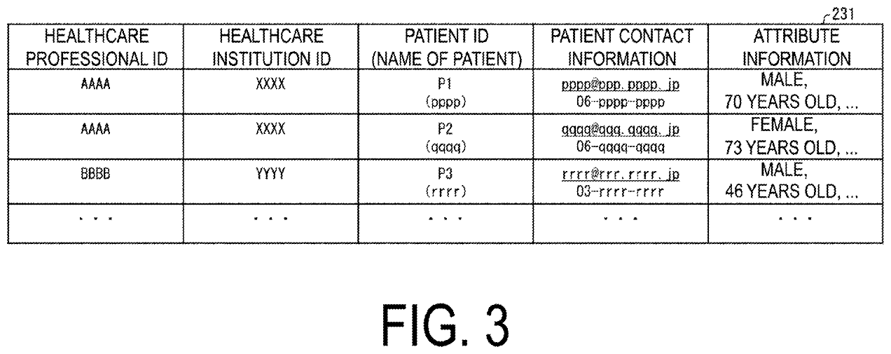

to FIGS. 3 to 5. FIG. 2 is a functional block diagram illustrating an example of a configuration of the server device 2. The server device 2 includes a control unit 20 corresponding to a central processing unit (CPU) included in the server device 2, and a storage unit 23 corresponding to a memory included in the server device 2. The server device 2 also includes a communication unit 24 that performs communication between the recommended exercise presentation device 1 and the communication terminal 4.

### Storage Unit 23

The storage unit 23 is a storage device in which various computer programs to be read by a control unit 20, data used in various kinds of processing executed by the control unit 20, and the like are stored. The storage unit 23 may store a registration information DB 231, a recommended exercise DB 232, and a web page DB 233.

### Registration Information DB 231

FIG. 3 is a diagram illustrating an example of a data structure of registration information 231. The registration information DB 231 includes information on each healthcare professional who has registered use of the exercise therapy support system 100 and the patient who are instructed to perform the exercise therapy by the healthcare professional.

As shown in FIG. 3, the registration information DB 231 may include the healthcare professional ID of each healthcare professional, the healthcare institution ID of the healthcare institution to which the healthcare professional belongs, the patient ID (and patient's name), patient contact information, and patient's attribute information.

The healthcare professional ID may be identification information unique to each healthcare professional that is assigned to each healthcare professional who has registered use of the exercise therapy support system 100, for example. The healthcare professional ID may be the name, an electronic mail address, or the like of the healthcare professional. The healthcare institution ID may be a code number unique to the healthcare institution to which the healthcare professional belongs. The patient ID may be identification information unique to each patient which is assigned by a healthcare professional (or the healthcare facility to which the healthcare professional belongs). The patient ID may be the name of the patient. The patient contact information may be contact information for transmitting various notifications from the server device 2 to the recommended exercise presentation device 1 used by the patient, and may be, for example, an e-mail address and/or a telephone number with which a short message service can be used. The attribute information may include age and sex of the patient. The attribute information may include a medical history of the patient and/or a period during which the patient received the exercise therapy in the past. The attribute information may include information indicating physical characteristics of the patient such as height and weight of the patient.

### Recommended Exercise DB 232

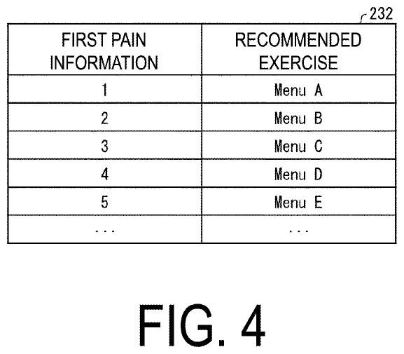

FIG. 4 is a diagram illustrating an example of a data structure of the recommended exercise DB 232. In the recommended exercise DB 232, a recommended exercise may be defined for each piece of first pain information. The first pain information may include at least one selected from the group consisting of a degree of pain in the target site, a type of the pain, and a combination thereof. For example, “1” to “5” and others of the first pain information shown in FIG. 4 may be numerical values when the degree of pain is divided into a plurality of stages (levels). For example, the first pain information “1” may mean that the degree of pain is the strongest, and the degree of pain may be indicated as having weaker intensities as the first pain information increases to “2”, “3”, and others. The first pain information may include “0” indicating that no pain is present in addition to “1” to “5” and others illustrated in FIG. 3.

Alternatively, the first pain information may be represented as a numerical value obtained by combining the degree of pain with the type of pain (e.g., pain, dull pain, colic, intermittent pain, or the like) and classifying the result. For example, the first pain information “1” may mean “pain” with a high degree of pain, and the first pain information “2” may mean “dull pain” with a low degree of pain. The first pain information is not limited to being represented by a number and may be represented in another form. For example, the first pain information may be expressed as “severe, moderate, and mild” or may be expressed as “symptom A, symptom B, and others”.

In the recommended exercise DB 232 shown in FIG. 4, the recommended exercise “Menu A” is associated with the first pain information “1”, and the recommended exercise “Menu B” is associated with the first pain information “2”. “Menu A” to “Menu E” may be an exercise program including one or more types of exercise.

The recommended exercise differs depending on whether the target site is a knee joint, a shoulder joint, or a hip joint, for example. The recommended exercise varies depending on, for example, a state such as presence or absence of swelling of the target site. Therefore, in the recommended exercise DB 232, the recommended exercise may be defined for each combination of information about the target site and the first pain information. Here, the “information about the target site” may be information indicating where the target site is (e.g., the name of the target site), or may include various pieces of information such as a degree of swelling of the target site, a history of exercise therapy conducted on the target site in the past, the medical history of the target site, and the period of pain in the target site.

In the recommended exercise DB 232, the recommended exercise is defined in accordance with the symptom of the target site of the patient in addition to the first pain information, and thus a recommended exercise determination unit 202 to be described below can determine a recommended exercise more suitable for the patient.

### Web Page DB 233

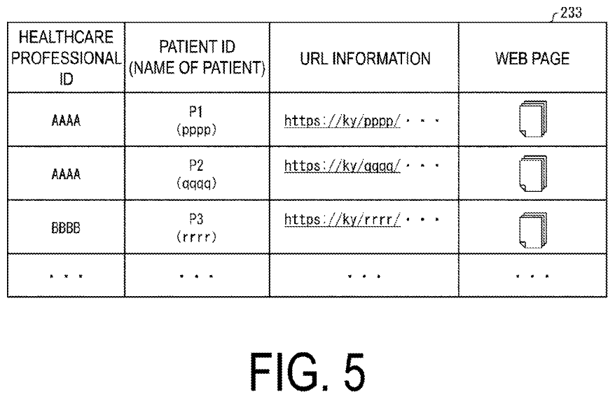

FIG. 5 is a diagram illustrating an example of a data structure of the web page DB 233. The web page DB 233 stores data of web pages generated by the server device 2 for each patient and Uniform Resource Locator (URL) information of each web page. As shown in FIG. 5, the data of each web page included in the web page DB 233 may be associated with the healthcare professional ID and the patient ID corresponding to the web page.

### Control Unit 20

The control unit 20 includes a user information acquisition unit 201, a recommended exercise determination unit 202, a period setting reception unit 203, a web page generation unit 204, and an access management unit 205, which are implemented by a CPU executing a predetermined computer program.

The user information acquisition unit 201 acquires patient information (user information) from the recommended exercise presentation device 1 or the communication terminal 4 of a healthcare professional. Here, the patient information may also include personal information such as the name and patient ID of the patient, address of the patient, age of the patient, the target site being treated, and severity of the symptom of the target site. The patient information may include effect information to be described below. After the user information acquisition unit 201 completes acquisition of the patient information, the server device 2 may transmit a second completion report indicating that the patient information has become browsable to the communication terminal 4 of the healthcare professional. For example, when the web page generation unit 204 to be described below generates a web page of the patient based on the patient information of the patient, the patient information may be browsable.

The recommended exercise determination unit 202 determines a recommended exercise suitable for the patient based on the first pain information of the patient. The recommended exercise determination unit 202 may determine the recommended exercise with reference to the recommended exercise DB 232. When the recommended exercise DB 232 is prepared for each target site, the recommended exercise determination unit 202 may determine the recommended exercise based on the first pain information and the information about the target site.

The period setting reception unit 203 receives the setting of the exercise period during which the patient is caused to perform the recommended exercise from the communication terminal 4 of the healthcare professional corresponding to the patient.

The web page generation unit 204 generates a web page based on the patient information including various kinds of information about the exercise therapy for the patient.

The web page generation unit 204 stores the generated web page in the web page DB 233. The web page generated by the web page generation unit 204 includes the patient ID, the recommended exercise determined based on the first pain information of the patient, and the exercise period set by the healthcare professional.

The web page generated by the web page generation unit 204 may include motion evaluation information obtained by evaluating whether a motion of the patient is appropriate as a motion for the recommended exercise presented to the patient based on detection information about the motion of the target site of the patient. The detection information may be acquired from the recommended exercise presentation device 1. Motion evaluation information may be, for example, “you've made a good motion!” or a message saying “you have a fast tempo”. As a result, the patient can ascertain whether he or she is making an appropriate motion.

The web page generated by the web page generation unit 204 may include information about an appropriate motion as a motion for the recommended exercise presented to the patient (e.g., link information with respect to a guidance moving image obtained by imaging a model of the recommended exercise). Accordingly, the patient can be supported to perform the recommended exercise correctly.

The access management unit 205 reads the web page of the patient from the web page DB 233 upon access from the recommended exercise presentation device 1 and access from the communication terminal 4.

### Recommended Exercise Presentation Device 1

The recommended exercise presentation device 1 is a computer used by a patient. The recommended exercise presentation device 1 may be a smartphone, a tablet terminal, a personal computer, or the like. Any known hardware and an OS of the computer are applicable. For example, by reading the recommended exercise presentation application 131 installed in the computer used by the patient from the storage unit 13 and executing the application, the computer can realize various functions as the recommended exercise presentation device 1.

The recommended exercise presentation device 1 may include, for example, an input unit 11 such as a keyboard, a touch panel, and/or a microphone, and a presentation unit 12 such as a monitor. The recommended exercise presentation device 1 receives input of first pain information about a degree of pain of a patient at a target site. The first pain information may be input based on decision of the patient himself or herself, or in response to advice or interview from healthcare professional. For example, the first pain information may be input by the healthcare professional corresponding to the patient operating the recommended exercise presentation device 1 used by the patient. The recommended exercise presentation device 1 may transmit the first pain information to the server device 2.

The recommended exercise presentation device 1 may access the server device 2 and cause the presentation unit 12 to display a patient-specific web page generated by the server device 2. The web page includes information about recommended exercise suitable for the patient, and information about an exercise period set by a healthcare professional. The patient can ascertain necessary information about his/her exercise therapy by browsing the web page. The recommended exercise presentation device 1 includes a communication unit 14 that performs communication with the server device 2. The recommended exercise presentation device 1 may include a detection unit 15 that detects a motion of a target site of the patient. The detection unit 15 may be various sensors (i.e., an acceleration sensor or the like) included in the recommended exercise presentation device 1. Alternatively, the detection unit 15 may be, for example, an imaging device (i.e., a video camera or the like) included in the recommended exercise presentation device 1.

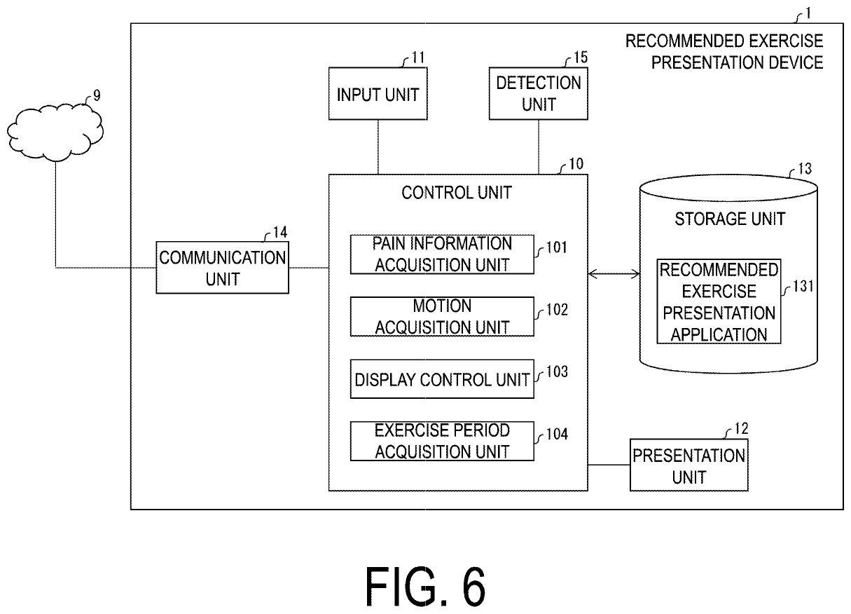

A configuration of the recommended exercise presentation device 1 will be described using FIG. 6. FIG. 6 is a functional block diagram illustrating an example of a configuration of the recommended exercise presentation device 1. The recommended exercise presentation device 1 includes a control unit 10 corresponding to a CPU included in the recommended exercise presentation device 1 and a storage unit 13 corresponding to a memory included in the recommended exercise presentation device 1.

### Storage Unit 13

The storage unit 13 is a storage device in which various computer programs to be read by the control unit 10, data used in various kinds of processing executed by the control unit 10, and the like are stored. The storage unit 23 may store the recommended exercise presentation application 131.

### Control Unit 10

The control unit 10 includes a pain information acquisition unit 101, a motion acquisition unit 102, and a display control unit 103 which are implemented by the CPU executing a predetermined computer program. The control unit 10 may further include an exercise period acquisition unit 104.

The pain information acquisition unit 101 acquires first pain information about the degree of pain of the patient who uses the recommended exercise presentation device 1 at the target site. The pain information acquisition unit 101 may be configured to acquire the first pain information input from the input unit 11. The acquired first pain information is transmitted to the server device 2 via the communication unit 14.

The motion acquisition unit 102 acquires detection information about a motion of the target site of the patient from the detection unit 15. The motion acquisition unit 102 may have an arbitrary image analysis function capable of analyzing motions of the patient captured in a video. In this case, the motion acquisition unit 102 acquires detection information from the image analysis result. The acquired detection information may be transmitted to the server device 2 via the communication unit 14.

The display control unit 103 causes the presentation unit 12 to display the web page generated by the server device 2. The web page includes information about recommended exercise for the patient who uses the recommended exercise presentation device 1. For this reason, if the web page is displayed on the presentation unit 12, the presentation unit 12 can present the recommended exercise determined based on the first pain information of the patient. Recommended exercise determined by the server device 2 may be determined based on the first pain information movement and information about the target site. For this reason, if the web page is displayed on the presentation unit 12, the presentation unit 12 can present the recommended exercise determined based on the first pain information of the patient and information about the target site. The web page generated by the server device 2 may include motion evaluation information. Thus, if the web page is displayed by the presentation unit 12, the presentation unit 12 can also function as a presentation unit that presents the motion evaluation information.

The exercise period acquisition unit 104 acquires an exercise period during which the patient is caused to perform the recommended exercise, which is set by the healthcare professional. The exercise period acquisition unit 104 may acquire a method related to the exercise period from the server device 2. For example, when the web page includes information about an exercise period in which the recommended exercise is to be continued, which is set by the healthcare professional corresponding to the patient, the exercise period acquisition unit 104 may acquire the information about the exercise period from the web page.

### Communication Terminal 4

The communication terminal 4 is a computer used by a healthcare professional corresponding to a patient (that is, a patient who uses the recommended exercise presentation device 1). In the present specification, “healthcare professional” may include doctors, nurses, physiotherapists, dieticians, and caregivers. The communication terminal 4 may be a smartphone, a tablet terminal, a personal computer, or the like. Any known hardware and an OS of the computer are applicable. The communication terminal 4 may include, for example, an input unit 41 such as a keyboard, a touch panel, and a microphone, and a display unit 42 such as a monitor.

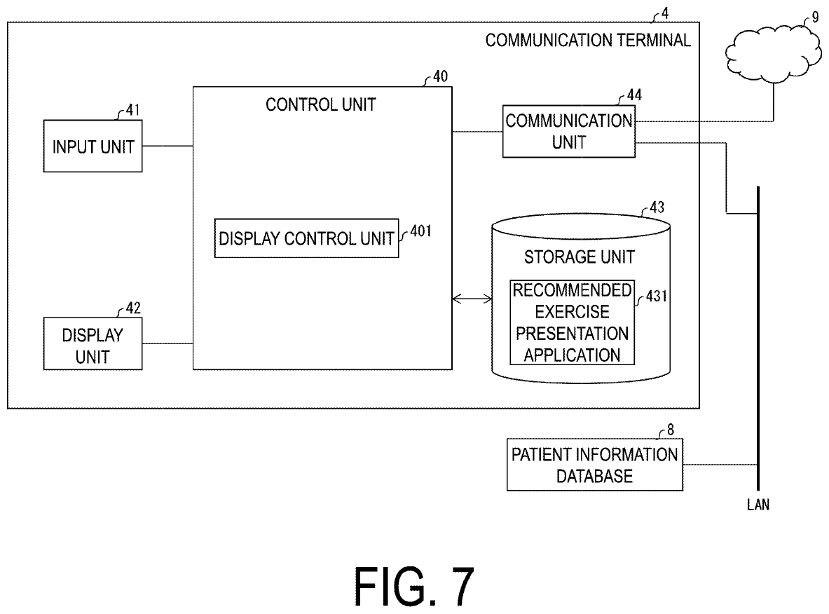

A configuration of the communication terminal 4 will be described using FIG. 7. FIG. 7 is a functional block diagram illustrating an example of a configuration of the communication terminal 4. The communication terminal 4 includes a control unit 40 corresponding to a CPU included in the communication terminal 4 and a storage unit 43 corresponding to a memory included in the communication terminal 4. The communication terminal 4 includes a communication unit 44 that performs communication with the server device 2.

The communication terminal 4 may be connected to a patient information database 8 storing electronic clinical record information and the like via a Local Area Network (LAN) arranged in a healthcare institution to which the healthcare professional using the communication terminal 4 belongs as illustrated in FIG. 7. In other words, the communication terminal 4 may be a computer used by a healthcare professional in the healthcare institution. In this case, the healthcare professional can check the information about the exercise therapy of the patient while referring to the electronic clinical record information of the patient, a medical image obtained by imaging the target site of the patient, and the like using the communication terminal 4.

### Storage Unit 43

The storage unit 43 is a storage device in which various computer programs to be read by the control unit 40, data used in various kinds of processing executed by the control unit 40, and the like are stored. The storage unit 43 may store the recommended exercise presentation application 431.

### Control Unit 40

The control unit 40 includes a display control unit 401 that is implemented by the CPU executing a predetermined computer program.

The display control unit 401 causes the display unit 42 to display a web page generated by the server device 2. The web page includes information about recommended exercise for the patient diagnosed by the healthcare professional using the communication terminal 4. Thus, the display unit 42 presents the patient with the recommended exercise determined based on the first pain information of the patient.

### Overview of Processing Executed by Exercise Therapy Support System 100

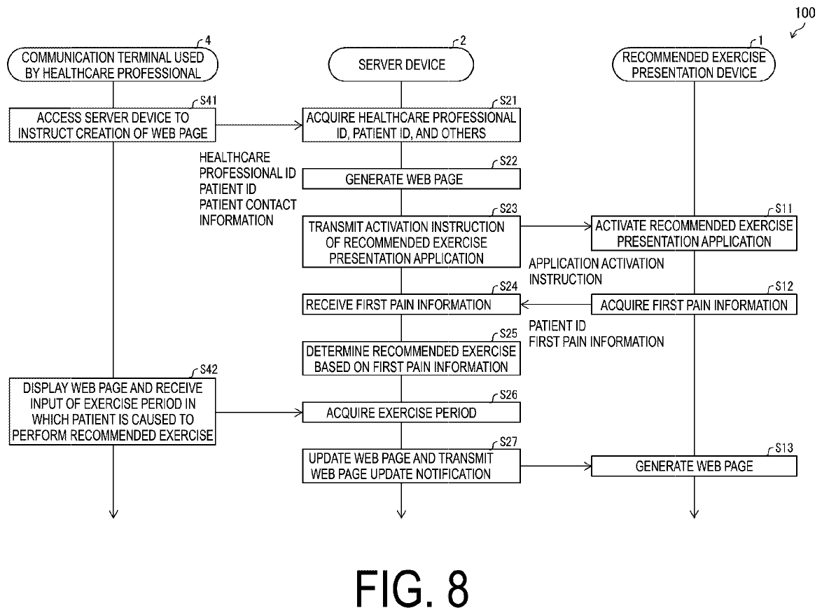

An overview of processing executed by the exercise therapy support system 100 will be described using FIG. 8. FIG. 8 is a sequence diagram illustrating an example of the flow of processing performed by the exercise therapy support system 100.

First, the communication terminal 4 accesses the server device 2 based on access information input by a healthcare professional. Then, the communication terminal 4 instructs creation of a web page in response to an input operation from the healthcare professional (step S41). This instruction may include the healthcare professional ID of the healthcare professional using the communication terminal 4, the patient ID of the patient who is caused to receive an exercise therapy, and patient contact information.

The server device 2 acquires the healthcare professional ID, the patient ID, and the like (step S21), and the web page generation unit 204 creates a web page dedicated to the patient (step S22). Thereafter, the web page generation unit 204 adds or updates various types of acquired information about the patient to the web page.

Next, the server device 2 transmits an activation instruction of the recommended exercise presentation application 131 to the recommended exercise presentation device 1 by using the patient contact information acquired in step S21 (step S23).

The recommended exercise presentation device 1 activates the recommended exercise presentation application 131 in response to an operation input of the patient who has confirmed the activation instruction from the server device 2 (step S11).

Subsequently, the display control unit 103 of the recommended exercise presentation device 1 causes the presentation unit 12 to display a screen for causing the patient to input information about the patient such as the patient ID and the first pain information. The pain information acquisition unit 101 acquires the first pain information input by the patient (step S12: pain information acquiring step). The acquired first pain information is transmitted to the server device 2 via the communication unit 14. The recommended exercise presentation device 1 transmits the patient ID together with the first pain information.

Next, the server device 2 receives the first pain information (step S24), and the recommended exercise determination unit 202 determines recommended exercise based on the received first pain information (step S25).

Meanwhile, the communication terminal 4 displays the web page generated by the server device 2 and receives an input of an exercise period during which the patient is caused to perform the recommended exercise (step S42). Then, the communication terminal 4 transmits information about the input exercise period to the server device 2.

Next, the server device 2 acquires the information about the exercise period from the communication terminal 4 (step S26), and the web page generation unit 204 adds the information about the exercise period to the web page of the patient to update the web page. The web page generation unit 204 may store the updated web page in the web page DB. The server device 2 transmits a notification for notifying the patient of the fact that the web page has been updated to the recommended exercise presentation device 1 (step S27).

Each time new information is added to the web page, or the web page is updated, the server device 2 may transmit an activation instruction of the recommended exercise presentation application 131 to the recommended exercise presentation device 1 as an alternative to the notification for notifying the patient of the fact that the web page has been updated. Accordingly, the exercise therapy support system 100 can cause the patient to quickly check the new information added to the web page dedicated to the patient and the updated information.

The recommended exercise presentation device 1 displays the updated web page on the presentation unit 12 in response to the operation input of the patient who has checked the notification from the server device 2 (step S13: presenting step).

As described above, the exercise therapy support system 100 determines the recommended exercise to be presented to the patient based on the first pain information about the target site of the patient. The recommended exercise presentation device 1 presents the recommended exercise determined in consideration of the state of the target site for each patient. Therefore, the patient can perform the recommended exercise suitable for the state of the target site with ease.

### First Variation

In step S12 of FIG. 8, the pain information acquisition unit 101 may further acquire attribute information including the age and/or sex of the patient himself/herself in addition to the first pain information input by the patient. The acquired first pain information and attribute information are transmitted to the server device 2 via the communication unit 14. The recommended exercise presentation device 1 may transmit the patient ID together with the first pain information and the attribute information.

In this case, the server device 2 receives the first pain information and the attribute information in step S24 of FIG. 8. Then, in step S24 of FIG. 8, the recommended exercise determination unit 202 may determine recommended exercise based on the received first pain information and attribute information.

When the registration information 231 also includes attribute information as illustrated in FIG. 3, the pain information acquisition unit 101 does not need to acquire the attribute information in step S12 of FIG. 8. In this case, the recommended exercise determination unit 202 determines the recommended exercise based on the received first pain information and the attribute information of the registration information 231 in step S24 of FIG. 8.

### Second Embodiment

Another embodiment of the present disclosure will be described below. For the sake of convenience of description, members having the same functions as those of the members described in the above-described embodiment are denoted by the same reference signs, and description thereof is not repeated.

A patient may be provided with the motion detection device 3 that detects motions of a target site of the patient together with the recommended exercise presentation application 131 from the healthcare professional. The motion detection device 3 may be a device that is attached to the body of the patient and outputs detection information to the recommended exercise presentation device 1. The motion detection device 3 may include various sensors (e.g., a motion sensor, a pressure sensor, and/or a myoelectric sensor). The motion detection device 3 may have the form of, for example, a supporter, a corset, a strap, clothing, a helmet (hat), socks, shoes, or the like that can be worn on the target site of the patient. The motion detection device 3 may be a rehabilitation device that can be attached to the target site of the patient. The motion detection device 3 may be a device capable of capturing a motion of the patient including the target site. In this case, the motion detection device 3 may include, for example, a video camera.

### Configuration of Exercise Therapy Support System 100a

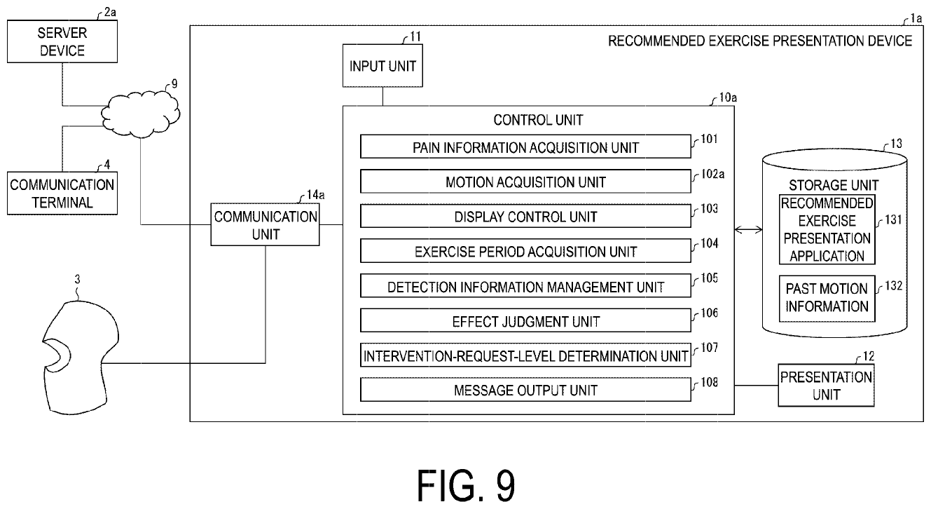

A configuration of an exercise therapy support system 100a will be described using FIG. 9. FIG. 9 is a diagram illustrating an example of a configuration of the exercise therapy support system 100a including a motion detection device 3. FIG. 9 illustrates the motion detection device 3 that can be worn on an elbow, a knee, or the like, as an example. The exercise therapy support system 100a includes a server device 2, a recommended exercise presentation device 1a used by a patient, the motion detection device 3 worn by the patient, and a communication terminal 4 used by a healthcare professional.

In the exercise therapy support system 100a, the recommended exercise presentation device 1a and the motion detection device 3 may be communicatively connected to each other. For example, the recommended exercise presentation device 1a and the motion detection device 3 may be connected by wireless communication or may be connected by wired communication.

In the exercise therapy support system 100a, a plurality of motion detection devices 3 may be provided for one recommended exercise presentation device 1. That is, in the exercise therapy support system 100a, the recommended exercise presentation device 1a may be configured to acquire detection information about motions of a plurality of target sites of the patient. When the target sites are the knees, for example, the recommended exercise presentation device 1a may acquire detection information from each of the motion detection device 3 for the left knee and the motion detection device 3 for the right knee.

### Recommended Exercise Presentation Device 1a

A configuration of the recommended exercise presentation device 1a will be described using FIG. 9. The recommended exercise presentation device 1a includes a control unit 10a corresponding to a CPU included in the recommended exercise presentation device 1a and a storage unit 13a corresponding to a memory included in the recommended exercise presentation device 1.

### Control Unit 10a

The control unit 10a includes a pain information acquisition unit 101, a motion acquisition unit 102a, a display control unit 103, and an exercise period acquisition unit 104 that are implemented by the CPU executing predetermined computer programs. The control unit 10a may further include at least one selected from the group consisting of a detection information management unit 105, an effect judgment unit 106, an intervention-request-level determination unit 107, and a message output unit 108.

The motion acquisition unit 102a acquires detection information about a motion of the target site of the patient from the motion detection device 3. The acquired detection information may be transmitted to the server device 2 via the communication unit 14. Accordingly, the recommended exercise presentation device 1a and the server device 2 can acquire more accurate detection information about the motion of the target site of the patient.

When the detection information cannot be acquired from the motion detection device 3, the motion acquisition unit 102a may cause the presentation unit 12 to present a notification indicating an error of the motion detection device 3.

The motion acquisition unit 102a may have a function of measuring the length of time during which no detection information is acquired from the motion detection device 3. In this case, the display control unit 103 may be configured to control the presentation unit 12 to present a message corresponding to the length of time during which the state in which the detection information is not acquired from the motion detection device 3 continues to the patient.

When the time in which a state of the motion detection device 3 acquiring no detection information continues for a first time (e.g., five minutes) or longer, the presentation unit 12 may prompt the patient to wear the motion detection device 3. For example, the presentation unit 12 may present a message or the like that prompts the patient to wear the motion detection device 3.

When a motion of the patient is not appropriate as a motion of the presented recommended exercise, the presentation unit 12 may present an appropriate motion as a motion of the recommended exercise presented to the patient. The presentation unit 12 may present a message explaining an appropriate motion as a motion of the recommended exercise or may present a video obtained by capturing the state of a third party (e.g., an instructor or the like) performing an appropriate motion as a motion of the recommended exercise.

When the time during which a state of the motion detection device 3 acquiring no detection information continues for a second time (e.g., six minutes) that is longer than the first time, the presentation unit 12 may present resolution information including a method of resolving the state to the patient. The presentation unit 12 may present, for example, a message prompting the patient to wear the motion detection device 3 again, a message prompting the patient to check his or her state wearing the motion detection device 3, or the like. A message or the like prompting the patient to wear the motion detection device 3 may be presented.

When the time during which the state of the motion detection device 3 acquiring no detection information continues for a third time (e.g., 10 minutes) that is longer than the second time, the presentation unit 12 may present motion evaluation information in which the motion of the user made while the state continued is regarded as appropriate or inappropriate as a motion of the presented recommended exercise.

In this manner, the recommended exercise presentation device 1a may notify the patient of the communication state between the recommended exercise presentation device 1a and the motion detection device 3 using the presentation unit 12. Thus, the exercise therapy support system 100a can appropriately support the exercise therapy of the patient using the recommended exercise presentation device 1a and the motion detection device 3.

The detection information management unit 105, the effect judgment unit 106, the intervention-request-level determination unit 107, and the message output unit 108 will be described in order below.

### Detection Information Management Unit 105

The detection information management unit 105 acquires first detection information indicating that a motion of the patient appropriate as a motion of the presented recommended exercise has been detected and second detection information indicating that a motion inappropriate as the motion of the presented recommended exercise has been detected. Then, the detection information management unit 105 may cause the storage unit 13a to store the first detection information and/or the second detection information as past motion information 132. Accordingly, the patient and/or the healthcare professional can confirm the appropriate motion and inappropriate motions of the recommended exercise performed by the patient later. Therefore, the patient can correct his/her own motion to be an appropriate motion as the presented recommended exercise and can perform more effective exercise therapy.

### Effect Judgment Unit 106

The effect judgment unit 106 compares the first pain information and the second pain information of the patient and judges a change in the degree of pain in the target site of the patient between before and after the recommended exercise is performed. Here, the second pain information is information about pain in the target site of the patient who has performed the recommended exercise for a first period. That is, the first pain information is information about pain before the patient performs the recommended exercise presented to the patient, whereas the second pain information is information about pain after the patient performs the recommended exercise for the first period. Information about pain may be, for example, the degree of pain (including the presence or absence of pain), the type of pain, or a combination thereof.

When the degree of pain in the target site of the patient is decreasing, the effect judgment unit 106 may determine to continue or change the recommended exercise for the user.

The effect judgment unit 106 may determine to change or stop the recommended exercise for the user when the degree of pain in the target site of the patient has been increasing or when the degree of pain in the target site of the user has not changed.

In addition, when the degree of pain in the target site of the patient has been increasing for a certain period of time, the effect judgment unit 106 may notify the healthcare professional corresponding to the patient of the effect information including the exercise history information about the history of the recommended exercise performed by the patient.

Here, the effect information may include at least one selected from the group consisting of the first pain information, the information about the recommended exercise presented to the patient by the recommended exercise presentation device 1, the second pain information, and the exercise history information. The effect information may include the intervention-request-level information indicating the level at which intervention is required, which is generated for the patient based on the comparison result obtained by comparing the first pain information and the second pain information of the patient. The intervention-request-level information will be described below.

The recommended exercise presentation device 1a may present the determination content by the effect judgment unit 106 to the presentation unit 12. The recommended exercise presentation device 1a may transmit the determination content by the effect judgment unit 106 to the server device 2. In this case, the web page generation unit 204 of the server device 2 may add the determination content by the effect judgment unit 106 acquired from the recommended exercise presentation device 1a to the web page of the patient.

The recommended exercise presentation device 1a may transmit the determination content by the effect judgment unit 106 to the communication terminal 4 or the server device 2. When the degree of pain in the target site of the patient has been increasing for a certain period of time, the recommended exercise presentation device 1a may transmit effect information including exercise history information about the history of the recommended exercise performed by the patient to the communication terminal 4 or the server device 2.

Since the recommended exercise presentation device 1a includes the effect judgment unit 106, a recommended exercise more suitable for the target site of the patient can be discovered at an early stage and the patient can be caused to perform a more effective exercise therapy.

Here, the second pain information is information about pain in the target site of the patient who has performed the recommended exercise for the first period. The first period may coincide with an exercise period set by the healthcare professional. In this case, the healthcare professional can decide whether to extend the exercise period of the exercise therapy of the patient based on how much the degree of pain in the target site of the patient has changed after the set exercise period.

### Intervention-Request-Level Determination Unit 107

The intervention-request-level determination unit 107 generates intervention-request-level information indicating a level at which intervention is required based on the comparison result obtained by comparing the first pain information and the second pain information.

The recommended exercise presentation device 1a may present the intervention-request-level information on the presentation unit 12. The recommended exercise presentation device 1a may transmit the intervention-request-level information to the server device 2. In this case, the web page generation unit 204 of the server device 2 may add the intervention-request-level information acquired from the recommended exercise presentation device 1a to the web page of the patient.

Since the recommended exercise presentation device 1a includes the intervention-request-level determination unit 107, the patient can be notified of the necessity of medical examination by a healthcare professional and/or a treatment other than the exercise therapy.

### Message Output Unit 108

When a patient who has not performed the recommended exercise satisfies the following condition during an exercise period in which the patient is caused to perform the recommended exercise, the message output unit 108 may output a message prompting the patient to perform the recommended exercise.

(Condition) Using a function other than the function of presenting the recommended exercise and the function of detecting a motion of the patient during the recommended exercise included in the recommended exercise presentation device 1a.

The message output unit 108 may output a message having a content corresponding to a usage state of a function other than presentation of the recommended exercise and detection of a motion during the recommended exercise.

The message output unit 108 may output a message having a content corresponding to the length of the use time of another function being used.

The message output unit 108 may output a message having a content corresponding to the timeslot in which the patient performed the recommended exercise in the past.

When the patient does not perform the recommended exercise for a predetermined time after a message is output, the message output unit 108 may output a message different from those messages.

The message output unit 108 may be, for example, a chatbot implemented by activating the recommended exercise presentation application 131. Here, the chatbot is a known communication function that can be mounted in the recommended exercise presentation device 1a. The message output unit 108 can prompt the patient to perform the recommended exercise by acting on the patient in a mode suitable for the patient's state.

For example, when all of the following (condition i) to (condition ii) are satisfied, the message output unit 108 may output a message prompting the patient to perform the recommended exercise. (Condition i) The recommended exercise presentation device 1a is activated, and the recommended exercise presentation application 131 is activated in the background. (Condition ii) The patient is using a function (e.g., a telephone, and the like) of the recommended exercise presentation device 1a other than the recommended exercise presentation application 131.

In addition, the message output unit 108 may output a message prompting the patient to perform the recommended exercise in the following states (1) to (3). The message output unit 108 may output the message by displaying a pop-up on the presentation unit 12 or may transmit the message using a social networking service (SNS) function and an e-mail function included in the recommended exercise presentation device 1a. Alternatively, the message output unit 108 may cause the message to be output using a sound output function included in the recommended exercise presentation device 1a.
(1) A state of the patient using the recommended exercise presentation device 1a to use an application which is different from the recommended exercise presentation application 131 and is other than a telephone or an e-mail application. However, the message output by the message output unit 108 may disturb the patient, depending on the attribute information of the application. For example, when the patient is using a cooking application, the patient is likely to be cooking, and thus outputting a message prompting the patient to perform the recommended exercise disturbs the patient. Thus, the message output unit 108 may determine whether to output a message prompting the patient to perform the recommended exercise in accordance with the attributes of the application that is different from the recommended exercise presentation application 131 and is other than a telephone or an email application.
(2) A state in which the recommended exercise presentation application 131 has been used for a certain period of time (e.g., 30 minutes) but no detection information has yet been acquired from the motion detection device 3.
(3) A state of the patient using an application different from the recommended exercise presentation application 131 in a timeslot in which the recommended exercise presentation application 131 has been used last time. For example, the message output unit 108 may output a message prompting the user to perform the recommended exercise in the timeslot in which the recommended exercise presentation application 131 is often used by the patient. When another application is used in the timeslot, the message output unit 108 proposes the start of the exercise therapy through a pop-up notification.

The message output unit 108 may change the content of the message to be output in accordance with the number of times that the message output by the message output unit 108 has been ignored (e.g., there has been no reply, or the patient has not been started the recommended exercise in response to the message). In this case, for example, when the recommended exercise of the patient is detected within a predetermined time (e.g., 15 minutes) according to the message output by the message output unit 108, the number of times that the message has been ignored is not counted.

The message output unit 108 may notify the communication terminal 4 of the healthcare professional corresponding to the patient of the situation of the patient based on the number of times the output message has been ignored and/or the number of times the recommended exercise presentation application 131 has been activated.

As described above, the message output unit 108 can prompt the user to receive the exercise therapy and give the user chances to continue the exercise therapy in various situations.

### Third Embodiment

Another embodiment of the present disclosure will be described below. For the sake of convenience of description, members having the same functions as those of the members described in the above-described embodiment are denoted by the same reference signs, and description thereof is not repeated.

The web page generation unit 204 of the server device 2 illustrated in FIG. 2 may be capable of generating a group web page in which a plurality of patients registered in the exercise therapy support system 100 or 100a are allowed to browse at least part of each other's patient information. The patient information that is not allowed to be browsed by each other may include personal information, for example, name of patients, patient IDs, and addresses of patients. The plurality of patients may be selected based on the patient information.

The web page generation unit 204 may select a target to disclose a created group web

page based on the patient information. The web page generation unit 204 may receive contributions of comments posted on a group web page from a plurality of patients belonging to the group that can browse the information.

The web page generation unit 204 may select a target to disclose the created group web pages based on the following information (a) to (c).
(a) Number of Days for Which Exercise Therapy is Continued In this case, the web page generation unit 204 may group patients having the same number of continuation days or may group patients such that the number of continuation days varies.
(b) Symptoms of Target Site In this case, the web page generation unit 204 may group patients having the same target sites and symptoms or may group patients such that target sites and severity vary.
(c) Address In this case, the web page generation unit 204 may group patients who visits the same hospital or may group patients living in close locations base on their addresses (e.g., living in the same area).

The web page generation unit 204 may include a suggestion for providing an opportunity to gather and perform exercise in the group web page. The suggestion may be, for example, a suggestion to exercise at an exercise facility close to the addresses of patients in the same group, or to walk on a recommended course.

By disclosing the group web page to a plurality of patients, the other patients in the same group can be encouraged to promote each of the patients to perform the exercise therapy and given an opportunity to continue the exercise therapy.

### Fourth Embodiment

Another embodiment of the present disclosure will be described below. For the sake of convenience of description, members having the same functions as those of the members described in the above-described embodiment are denoted by the same reference signs, and description thereof is not repeated.

In the exercise therapy support system 100 or 100a, the recommended exercise (see FIG. 4) may be made into a library and may include basic motions and/or a specialized exercise specialized in a symptom of a target site.

In the exercise therapy support system 100 or 100a, a graph may be created so that the number of days on which the patient performed the recommended exercise and/or the effect of the recommended exercise can be checked as a change over time. The patient and healthcare professional may be able to check the graph on a web page. As a result, the exercise therapy support systems 100 and 100a can give the patient encouragement to continuously perform the exercise therapy. Here, the server device 2 may create the graph automatically, or the server device 2 may acquire a graph created by a healthcare professional.

In the exercise therapy support system 100 or 100a, elements of a game may be incorporated to help patients continuously perform recommended exercise. For example, in the exercise therapy support system 100 or 100a, a configuration in which points are given as a “reward” every time the number of times the patient performs the recommended exercise exceeds a predetermined number of times may be adopted. In the exercise therapy support systems 100 and 100a, the number of times of recommended exercise performed may be ranked and displayed to motivate the patient. In the exercise therapy support systems 100 and 100a, avatars may be prepared for each of patients, and the avatars may become healthier or more grow as the number of times that the patient performed the recommended exercise increases.

As described above, the exercise therapy support systems 100 and 100a can employ various mechanisms for allowing patients to continuously perform recommended exercise.

The exercise therapy support systems 100 and 100a may be provided with a unit for a healthcare professional corresponding to a patient to notify the patient of information about the symptom of the target site of the patient. Examples of the notification from the healthcare professional to the patient include a notification indicating what happens if the symptom of the target site of the patient becomes worse and a notification indicating the importance of weight reduction (dietary restriction) to improve the symptom.

### Supplementary Note

The configurations described in the above-described embodiments are examples, and the roles of the server device 2, the recommended exercise presentation devices 1 and 1a, and the communication terminal 4 in the exercise therapy support systems 100 and 100a are not limited thereto. For example, the recommended exercise presentation devices 1 and 1a may include each function of the control unit 20 of the server device 2 and each piece of data of the storage unit 23 illustrated in FIG. 2. Alternatively, the communication terminal 4 may include each function of the control unit 20 of the server device 2 and each piece of data of the storage unit 23 illustrated in FIG. 2. In other words, in the exercise therapy support systems 100 and 100a, the recommended exercise presentation devices 1 and 1a, or the communication terminal 4 have the configuration to serve as the server device 2.

### Implementation Example by Software

Functions of the recommended exercise presentation devices 1 and 1a, the server device 2, and the communication terminal 4 (hereinafter referred to as “each of the devices”) can be implemented by a program for causing a computer to function as each device and for causing the computer to function as each control block of each of the devices (particularly, each unit included in the control units 10, 20, and 40).

In this case, each of the devices includes a computer including at least one control device (e.g., a processor) and at least one storage device (e.g., a memory) as hardware for executing the program. By executing the program by the control device and the storage device, the functions described in the embodiments are implemented.

The program may be recorded on one or more computer-readable non-transitory recording media. The recording media may or may not be included in each of the devices. In the latter case, the program may be supplied to each of the devices via any wired or wireless transmission medium.

Some or all of the functions of the control blocks can be implemented by logic circuits. For example, an integrated circuit in which logic circuits functioning as the control blocks are formed is also included in the scope of the present disclosure. In addition to this, for example, a quantum computer can implement the functions of the control blocks.

The invention according to the present disclosure has been described above based on the drawings and the examples. However, the invention according to the present disclosure is not limited to each embodiment described above. That is, the invention according to the present disclosure may be varied in diverse manners within the scope described in the present disclosure, and an embodiment obtained by appropriately combining technical means disclosed in different embodiments is also included in the technical scope of the invention according to the present disclosure. That is, note that one skilled in the art can easily make various variations or modifications based on the present disclosure. Also note that these variations or modifications are included in the scope of the present disclosure.

### REFERENCE SIGNS

1, 1a Recommended exercise presentation device
2 Server device
3 Motion detection device
4 Communication terminal
12 Presentation unit
15 Detection unit
100, 100a Exercise therapy support system
101 Pain information acquisition unit
102, 102a Motion acquisition unit
104 Exercise period acquisition unit
105 Detection information management unit
106 Effect judgment unit
107 Intervention-request-level determination unit
108 Message output unit
201 User information acquisition unit
203 Period setting reception unit
204 Web page generation unit (group page generation unit)
S12 Pain information acquiring step
S13 Presenting step

1. A recommended exercise presentation device comprising:
a pain information acquisition unit configured to acquire first pain information about pain in a target site of a user; and
a presentation unit configured to present, to the user, recommended exercise for the user, as determined based on the first pain information.

2. The recommended exercise presentation device according to claim 1,
wherein the first pain information comprises at least one selected from the group consisting of a degree of pain in the target site, a type of pain, and a combination of the degree of pain and the type of pain.

3. The recommended exercise presentation device according to claim 1,
wherein the presentation unit presents, to the user, the recommended exercise as determined based on the first pain information and information about the target site.

4. The recommended exercise presentation device according to claim 1, further comprising an exercise period acquisition unit configured to acquire an exercise period for the user to perform the recommended exercise, the exercise period being set by a healthcare professional.

5. The recommended exercise presentation device according to claim 1, comprising
a motion acquisition unit configured to acquire detection information about a motion of the target site of the user,
wherein the presentation unit presents motion evaluation information obtained by evaluating, based on the detection information, whether a motion of the user is appropriate as a motion of the presented recommended exercise.

6. The recommended exercise presentation device according to claim 5,
wherein the motion acquisition unit acquires the detection information from a motion detection device configured to acquire the detection information about the motion of the target site of the user.

7-8. (canceled)

9. The recommended exercise presentation device according to claim 5,
wherein when the motion of the user is inappropriate as the motion of the presented recommended exercise, the presentation unit presents an appropriate motion appropriate as the motion of the presented recommended exercise to the user.

10-13. (canceled)

14. The recommended exercise presentation device according to claim 5,
wherein the pain information acquisition unit acquires second pain information about pain in the target site of the user who has performed the recommended exercise for a first period, before and/or after the user performs the recommended exercise a next time, and
the recommended exercise presentation device further comprising,
an effect judgment unit configured to compare the first pain information of the user with the second pain information of the user after the user performs the recommended exercise in the first period, and judge a change in a degree of pain in the target site of the user before and after the user performs the recommended exercise.

15-20. (canceled)

21. The recommended exercise presentation device according to claim 5, further comprising
a message output unit configured to output a message, in an exercise period where the user is expected to perform the recommended exercise, with content prompting the user to perform the recommended exercise when the user who has not performed the recommended exercise is using a different function other than presentation of the recommended exercise and a detection of a motion during the recommended exercise.

22-26. (canceled)

27. A server device configured to communicate with the recommended exercise presentation device according to claim 1 and a communication terminal used by a healthcare professional corresponding to at least one user of the recommended exercise presentation device, the server device comprising:
a period setting reception unit configured to receive, from the communication terminal used by the healthcare professional, a setting of an exercise period where the at least one user is prompted to perform the recommended exercise; and
a user information acquisition unit configured to acquire user information about the at least one user from the recommended exercise presentation device,
wherein the user information comprises effect information comprising at least one selected from the group consisting of the first pain information, information about the recommended exercise presented by the recommended exercise presentation device, second pain information about pain in the target site of the at least one user who has performed the recommended exercise for a first period, and exercise history information indicating a history of the recommended exercise that has been performed by the at least one user.

28. The server device according to claim 27,
wherein the effect information comprises intervention-request-level information indicating a level where intervention is requested, the intervention-request-level information being generated for the at least one user based on a comparison result obtained by comparing the first pain information of the at least one user with the second pain information of the at least one user who has performed the recommended exercise for the first period.

29. The server device according to claim 27,
wherein a first completion report indicating that the exercise period has ended is transmitted to the communication terminal used by the healthcare professional at the end of the exercise period.

30. The server device according to claim 27,
wherein a second completion report indicating that the user information has become browsable is transmitted to the communication terminal used by the healthcare professional after the user information acquisition unit completes acquisition of the user information.

31. The server device according to claim 27,
wherein whenever the at least one user has performed the recommended exercise, a report regarding a fact that the at least one user has performed the recommended exercise is transmitted to the communication terminal used by the healthcare professional.

32. The server device according to claim 27,
wherein when a period where the at least one user does not perform the recommended exercise is equal to or longer than a second period, an exercise suspension report regarding a fact that the period where the at least one user does not perform the recommended exercise is equal to or longer than the second period is transmitted to the communication terminal used by the healthcare professional.

33-34. (canceled)

35. An exercise therapy support system comprising:
the recommended exercise presentation device according to claim 1,
a communication terminal used by a healthcare professional corresponding to a user of the recommended exercise presentation device; and
a server device comprising:
a period setting reception unit configured to receive, from the communication terminal used by the healthcare professional, a setting of an exercise period where the user is prompted to perform the recommended exercise; and
a user information acquisition unit configured to acquire user information about the user from the recommended exercise presentation device,
wherein the user information comprises effect information comprising at least one selected from the group consisting of the first pain information, information about the recommended exercise presented by the recommended exercise presentation device, second pain information about pain in the target site of the user who has performed the recommended exercise for a first period, and exercise history information indicating a history of the recommended exercise that has been performed by the user.

36. An exercise therapy support system comprising:
the recommended exercise presentation device according to claim 1;
a motion detection device configured to acquire detection information about a motion of the target site of the user;
a communication terminal used by a healthcare professional corresponding to a user of the recommended exercise presentation device; and
a server device comprising:
a period setting reception unit configured to receive, from the communication terminal used by the healthcare professional, a setting of an exercise period where the user is prompted to perform the recommended exercise; and
a user information acquisition unit configured to acquire user information about the user from the recommended exercise presentation device,
wherein the user information comprises effect information comprising at least one selected from the group consisting of the first pain information, information about the recommended exercise presented by the recommended exercise presentation device, second pain information about pain in the target site of the user who has performed the recommended exercise for a first period, and exercise history information indicating a history of the recommended exercise that has been performed by the user.

37. A recommended exercise presentation method comprising:
acquiring first pain information about pain in a target site of a user; and
presenting, to the user, recommended exercise for the user, as determined based on the first pain information.

38. A non-transitory computer-readable recording medium that stores a control program for causing an electronic device to:
function as the recommended exercise presentation device according to claim 1, and
function as the pain information acquisition unit and the presentation unit.

39. A non-transitory computer-readable recording medium that stores a control program for causing an electronic device to:
function as the server device according to claim 27, and
function as the period setting reception unit and the user information acquisition unit.
